(19) United States
(12) Patent Application Publication
(10) Pub. No.: US 2019/0121506 A1
(43) Pub. Date: Apr. 25, 2019

(54) USER INTERFACE FOR TIME-SENSITIVE EVENT ENTRY IN ANALYTE MONITORING SYSTEM

(71) Applicant: Senseonics, Incorporated, Germantown, MD (US)

(72) Inventor: Rober Matikyan, Germantown, MD (US)

(73) Assignee: Senseonics, Incorporated, Germantown, MD (US)

(21) Appl. No.: 16/230,448

(22) Filed: Dec. 21, 2018

Related U.S. Application Data
(63) Continuation-in-part of application No. 15/167,559, filed on May 27, 2016.
(60) Provisional application No. 62/610,668, filed on Dec. 27, 2017.
(60) Provisional application No. 62/166,972, filed on May 27, 2015.

(51) Int. Cl. G06F 3/0482; A61B 5/00; G16H 20/60; A61B 5/145; G06F 3/01

(52) U.S. Cl. CPC G06F 3/0482; A61B 5/742; G06F 3/017; A61B 5/14532; G16H 20/60

(57) ABSTRACT

An analyte monitoring system may include a display device configured to (i) receive and display analyte concentrations, (ii) select one of a plurality of meal types as a default meal type based on the current time of day, and (iii) display a meal event entry screen that includes a meal type selectable element for specifying a type of meal. The meal type selectable element may be initially set to the selected default meal type, and the meal type selectable element may be capable of being set to a different one of the plurality of meal types. The display device may enable a user to adjust default time intervals for selecting the default meal type, and the display device may display an event log screen that includes an add event selectable element for adding one or more events including one or more meal events based on the adjusted default time intervals.

### CROSS-REFERENCE TO RELATED APPLICATIONS

The present application claims the benefit of priority to U.S. Provisional Application Ser. No. 62/610,668, filed on Dec. 27, 2017, which is incorporated herein by reference in its entirety. The present application is also a continuation-in-part of U.S. patent application Ser. No. 15/167,559, filed May 27, 2016, which claims priority to U.S. Provisional Patent Application Ser. No. 62/166,972, filed May 27, 2015, which are incorporated by reference herein in their entireties.

### BACKGROUND

### Field of Invention

Aspects of the present invention relate to systems and methods for analyte monitoring. Specifically, aspects of the present invention may relate to a user interface for time-sensitive event entry in an analyte monitoring system.

### Discussion of the Background

The prevalence of diabetes mellitus continues to increase in industrialized countries, and projections suggest that this figure will rise to 4.4% of the global population (366 million individuals) by the year 2030. Glycemic control is a key determinant of long-term outcomes in patients with diabetes, and poor glycemic control is associated with retinopathy, nephropathy and an increased risk of myocardial infarction, cerebrovascular accident, and peripheral vascular disease requiring limb amputation. Despite the development of new insulins and other classes of antidiabetic therapy, roughly half of all patients with diabetes do not achieve recommended target hemoglobin A1c (HbA1c) levels <7.0%.

Frequent self-monitoring of blood glucose (SMBG) is necessary to achieve tight glycemic control in patients with diabetes mellitus, particularly for those requiring insulin therapy. However, current blood (finger-stick) glucose tests are burdensome, and, even in structured clinical studies, patient adherence to the recommended frequency of SMBG decreases substantially over time. Moreover, finger-stick measurements only provide information about a single point in time and do not yield information regarding intraday fluctuations in blood glucose levels that may more closely correlate with some clinical outcomes.

Continuous glucose monitors (CGMs) have been developed in an effort to overcome the limitations of finger-stick SMBG and thereby help improve patient outcomes. These systems enable increased frequency of glucose measurements and a better characterization of dynamic glucose fluctuations, including episodes of unrealized hypoglycemia. Furthermore, integration of CGMs with automated insulin pumps allows for establishment of a closed-loop “artificial pancreas” system to more closely approximate physiologic insulin delivery and to improve adherence.

Monitoring real-time analyte measurements from a living body via wireless analyte monitoring sensor(s) may provide numerous health and research benefits. There is a need to enhance such analyte monitoring systems via innovations comprising, but not limited to, a user interface that enables a user to interact efficiently with an analyte monitoring system.

### SUMMARY

One aspect of the invention may provide an analyte monitoring system including an analyte sensor, a transceiver, and a display device. The transceiver may be configured to (i) receive measurement information from the analyte sensor, (ii) calculate one or more analyte concentrations using at least the received measurement information, and (iii) convey the calculated one or more analyte concentrations. The display device may be configured to (i) receive the one or more analyte concentrations from the transceiver, (ii) display the received one or more analyte concentrations, (iii) select one of a plurality of meal types as a default meal type based on the current time of day, and (iv) display a meal event entry screen that includes a meal type selectable element for specifying a type of meal. The meal type selectable element may be initially set to the selected default meal type, and the meal type selectable element may be capable of being set to a different one of the plurality of meal types.

In some embodiments, the display device may be configured to display a meal event entered using the meal event entry screen with the one or more analyte concentrations.

Another aspect of the invention may provide an analyte monitoring system including an analyte sensor and a transceiver. The transceiver may be configured to (i) receive measurement information from the analyte sensor, (ii) calculate one or more analyte concentrations using at least the received measurement information, (iii) display the calculated one or more analyte concentrations, (iv) select one of a plurality of meal types as a default meal type based on the current time of day, and (v) display a meal event entry screen that includes a meal type selectable element for specifying a type of meal. The meal type selectable element may be initially set to the selected default meal type, and the meal type selectable element may be capable of being set to a different one of the plurality of meal types.

In some embodiments, the transceiver may be configured to display a meal event entered using the meal event entry screen with the one or more analyte concentrations.

Yet another aspect of the invention may provide a display device including a transceiver, a user interface, and a computer. The transceiver interface device may be configured to receive one or more analyte concentrations from a transceiver. The computer may include a non-transitory memory and a processor. The computer may be configured to cause the user interface to display the one or more received analyte concentrations. The computer may be configured to select one of a plurality of meal types as a default meal type based on the current time of day. The computer may be configured to cause the user interface to display a meal event entry screen that includes a meal type selectable element for specifying a type of meal. The meal type selectable element may be initially set to the selected default meal type, and the meal type selectable element may be capable of being set to a different one of the plurality of meal types.

Still another aspect of the invention may provide a transceiver including a sensor interface device, a user interface, and a computer. The sensor interface device may be configured to receive sensor data from an analyte sensor. The computer may include a non-transitory memory and a processor. The computer may be configured to calculate one or more analyte concentrations using at least the received measurement information. The computer may be configured to cause the user interface to display the one or more calculated analyte concentrations. The computer may be configured to select one of a plurality of meal types as a default meal type based on the current time of day. The computer may be configured to cause the user interface to display a meal event entry screen that includes a meal type selectable element for specifying a type of meal. The meal type selectable element may be initially set to the selected default meal type, and the meal type selectable element may be capable of being set to a different one of the plurality of meal types.

In some embodiments, the computer may be configured to cause the user interface to display a meal event entered using the meal event entry screen with the one or more analyte concentrations. In some embodiments, the plurality of meal types may include breakfast, lunch, and dinner. In some embodiments, selecting the default meal type may include comparing the current time of day to time intervals corresponding to the meal types, and the selected default meal type may be the meal type having the time interval into which the current time of day falls. In some embodiments, the time intervals may be configured to be adjustable by a user of the display device or the transceiver.

Another aspect of the invention may provide an analyte monitoring system including an analyte sensor, a transceiver, and a display device. The transceiver may be configured to (i) receive measurement information from the analyte sensor, (ii) calculate one or more analyte concentrations using at least the received measurement information, and (iii) convey the calculated one or more analyte concentrations. The display device may be configured to (i) receive the one or more analyte concentrations from the transceiver, (ii) display the one or more received analyte concentrations, (iii) enable a user to adjust default time intervals for meal types of a plurality of meal types, and (iv) display an event log screen that includes an add event selectable element for adding one or more events including one or more meal events based on the adjusted default time intervals.

Still another aspect of the invention may provide a display device including a transceiver interface device, a user interface, and a computer. The transceiver interface device may be configured to receive one or more analyte concentrations from a transceiver. The computer may include a non-transitory memory and a processor. The computer may be configured to cause the user interface to display the one or more received analyte concentrations. The computer may be configured to cause the user interface to enable a user to adjust default time intervals for meal types of a plurality of meal types. The computer may be configured to cause the user interface to display an event log screen that includes an add event selectable element for adding one or more events including one or more meal events based on the adjusted default time intervals.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a schematic view illustrating an analyte monitoring system embodying aspects of the present invention.

FIG. 2 illustrates a block diagram of a display device of the analyte monitoring system according to some embodiments.

FIG. 3 illustrates a block diagram of a computer of the display device of the analyte monitoring system according to some embodiments.

FIG. 4 illustrates a non-limiting example of a home screen illustrative display of a medical mobile application in accordance with aspects of various embodiments of the present invention.

FIG. 5 illustrates a non-limiting example of a event screen display of a medical mobile application in accordance with aspects of various embodiments of the present invention.

FIG. 6 illustrates a non-limiting example of a meal event entry screen displayed on a display of a user interface of a display device in accordance with aspects of various embodiments of the present invention.

FIG. 7 illustrates a non-example of a menu navigational bar screen display of a medical mobile application in accordance with aspects of various embodiments of the present invention.

### DETAILED DESCRIPTION OF PREFERRED EMBODIMENTS

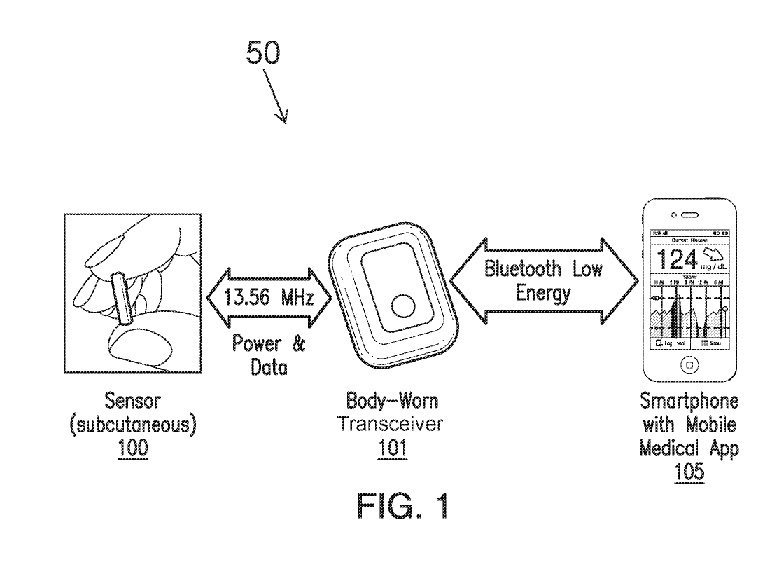

FIG. 1 is a schematic view of an exemplary analyte monitoring system 50 embodying aspects of the present invention. The analyte monitoring system 50 may be a continuous analyte monitoring system (e.g., a continuous glucose monitoring system). In some embodiments, the analyte monitoring system 50 may include one or more of an analyte sensor 100, a transceiver 101, and a display device 105. In some embodiments, the sensor 100 may be small, fully subcutaneously implantable sensor. However, this is not required, and, in some alternative embodiments, the sensor 100 may be a partially implantable (e.g., transcutaneous) sensor or a fully external sensor. In some embodiments, the transceiver 101 may be an externally worn transceiver (e.g., attached via an armband, wristband, waistband, or adhesive patch). In some embodiments, the transceiver 101 may communicate with the sensor to initiate and receive one or more sensor measurements via a wireless connection (e.g., via near field communication (NFC)) or a wired connection. In some embodiments, the sensor measurements may include one or more light measurements and/or one or more temperature measurements. In some embodiments, the one or more sensor measurements may be indicative of an amount or concentration of an analyte in a medium (e.g., interstitial fluid) of a living animal (e.g., a living human). In some non-limiting embodiments, the transceiver 101 may calculate one or more analyte concentrations using at least the received sensor measurements. In some embodiments, the transceiver 101 may communicate information (e.g., one or more analyte concentrations) wirelessly (e.g., via a Bluetooth™ communication standard such as, for example and without limitation Bluetooth Low Energy) to a mobile medical application (MMA) running on a display device 105 (e.g., a smartphone or tablet). In some embodiments, the MMA may additionally or alternatively receive the information receive the information from the transceiver 101 through a wired connection (e.g., using a Universal Serial Bus (USB)) port. In some embodiments, the analyte monitoring system 50 may include a web interface for plotting and sharing of the received information.

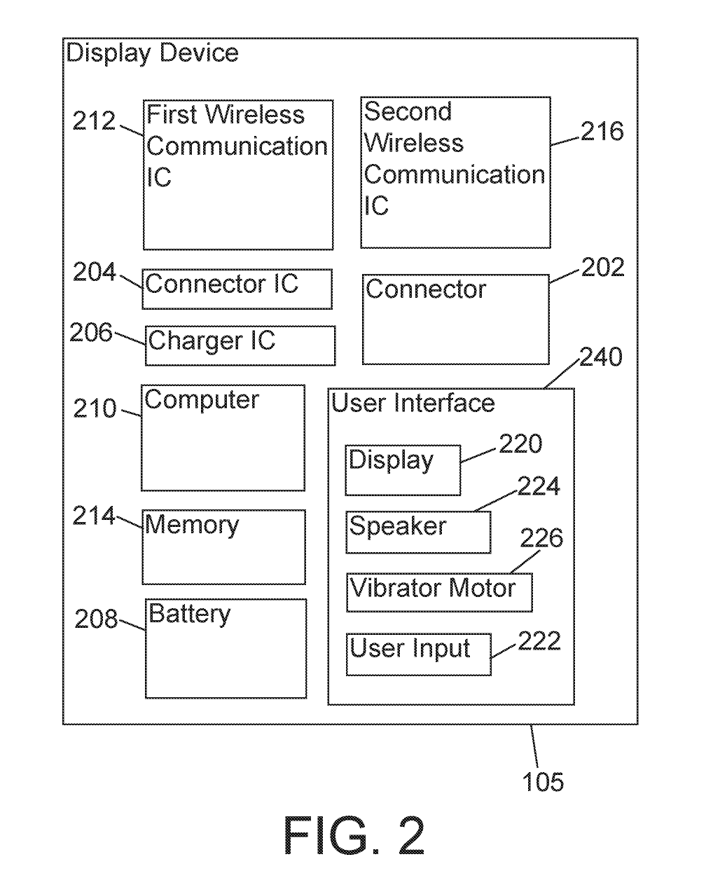

FIG. 2 is a block diagram of a non-limiting embodiment of the display device 105 of the analyte monitoring system 50. As shown in FIG. 2, in some embodiments, the display device 105 may include one or more of a connector 202, a connector integrated circuit (IC) 204, a charger IC 206, a battery 208, a computer 210, a first wireless communication IC 212, a memory 214, a second wireless communication IC 216, and a user interface 240.

In some embodiments in which the display device 105 includes the connector 202, the connector 202 may be, for example and without limitation, a Micro-Universal Serial Bus (USB) connector. The connector 202 may enable a wired connection to an external device, such as a personal computer or transceiver 101. The display device 105 may exchange data to and from the external device through the connector 202 and/or may receive power through the connector 202. In some embodiments, the connector IC 204 may be, for example and without limitation, a USB-IC, which may control transmission and receipt of data through the connector 202.

In some embodiments in which the display device 105 includes the charger IC 206, the charger IC 206 may receive power via the connector 202 and charge the battery 208. In some non-limiting embodiments, the battery 208 may be, for example and without limitation, a lithium-polymer battery. In some embodiments, the battery 208 may be rechargeable, may have a short recharge duration, and/or may have a small size.

In some embodiments, the display device 105 may include one or more connectors and/or one or more connector ICs in addition to (or as an alternative to) connector 202 and connector IC 204. For example, in some alternative embodiments, the display device 105 may include a spring-based connector (e.g., Pogo pin connector) in addition to (or as an alternative to) connector 202, and the display device 105 may use a connection established via the spring-based connector for wired communication to a personal computer or the transceiver 101 and/or to receive power, which may be used, for example, to charge the battery 208.

In some embodiments in which the display device 105 includes the first wireless communication IC 212, the first wireless communication IC 212 may enable wireless communication with one or more external devices, such as, for example, one or more personal computers, one or more transceivers 101, and/or one or more other display devices 105. In some non-limiting embodiments, the first wireless communication IC 212 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some non-limiting embodiments, the first wireless communication IC 212 may be configured to wirelessly transmit data at a frequency greater than 1 gigahertz (e.g., 2.4 or 5 GHz). In some embodiments, the first wireless communication IC 212 may include an antenna (e.g., a Bluetooth antenna). In some non-limiting embodiments, the antenna of the first wireless communication IC 212 may be entirely contained within a housing of the display device 105. However, this is not required, and, in alternative embodiments, all or a portion of the antenna of the first wireless communication IC 212 may be external to the display device housing.

In some embodiments, the display device 105 may include a transceiver interface device, which may enable communication by the display device 105 with one or more transceivers 101. In some embodiments, the transceiver interface device may include the antenna of the first wireless communication IC 212 and/or the connector 202. In some non-limiting embodiments, the transceiver interface device may additionally or alternatively include the first wireless communication IC 212 and/or the connector IC 204.

In some embodiments in which the display device 105 includes the second wireless communication IC 216, the second wireless communication IC 216 may enable the display device 105 to communicate with one or more remote devices (e.g., smartphones, servers, and/or personal computers) via wireless local area networks (e.g., Wi-Fi), cellular networks, and/or the Internet. In some non-limiting embodiments, the second wireless communication IC 216 may employ one or more wireless communication standards to wirelessly transmit data. In some embodiments, the second wireless communication IC 216 may include one or more antennas (e.g., a Wi-Fi antenna and/or one or more cellular antennas). In some non-limiting embodiments, the one or more antennas of the second wireless communication IC 216 may be entirely contained within a housing of the display device 105. However, this is not required, and, in alternative embodiments, all or a portion of the one or more antennas of the second wireless communication IC 216 may be external to the display device housing.

In some embodiments in which the display device 105 includes the memory 214, the memory 214 may be non-volatile and/or capable of being electronically erased and/or rewritten. In some embodiments, the memory 214 may be, for example and without limitations a Flash memory.

In some embodiments in which the display device 105 includes the computer 210, the computer 210 may control the overall operation of the display device 105. For example, the computer 210 may control the connector IC 204, the first wireless communication IC 212, and/or the second wireless communication IC 216 to transmit data via wired or wireless communication. The computer 210 may additionally or alternatively control processing of received data (e.g., analyte monitoring data received from the transceiver 101).

In some embodiments in which the display device 105 includes the user interface 240, the user interface 240 may include one or more of a display 220 and a user input 222. In some embodiments, the display 220 may be a liquid crystal display (LCD) and/or light emitting diode (LED) display. In some non-limiting embodiments, the user input 222 may include one or more buttons, a keyboard, a keypad, and/or a touchscreen. In some embodiments, the computer 210 may control the display 220 to display data (e.g., analyte concentration values, analyte trend information, alerts, alarms, and/or notifications). In some embodiments, the user interface 240 may include one or more of a speaker 224 (e.g., a beeper) and a vibration motor 226, which may be activated, for example, in the event that a condition (e.g., a hypoglycemic or hyperglycemic condition) is met.

In some embodiments, the computer 210 may execute a mobile medical application (MMA). In some embodiments, the display device 105 may receive analyte monitoring data from the transceiver 101. The received analyte monitoring data may include one or more analyte concentrations, one or more analyte concentrations trends, and/or one or more sensor measurements. The received analyte monitoring data may additionally or alternatively include alarms, alerts, and/or notifications. The MMA may display some or all of the received analyte monitoring data on the display 220 of the display device 105.

In some embodiments, the analyte monitoring system 50 may calibrate the conversion of raw sensor measurements to analyte concentrations. In some embodiments, the calibration may be performed approximately periodically (e.g., every 12 or 24 hours). In some embodiments, the calibration may be performed using one or more reference measurements (e.g., one or more self-monitoring blood glucose (SMBG) measurements). In some embodiments, the reference measurements may be entered into the analyte monitoring system 50 using the user interface 240 of the display device 105. In some embodiments, the display device 105 may convey one or more references measurements to the transceiver 101, and the transceiver 101 may use the one or more received reference measurements to perform the calibration.

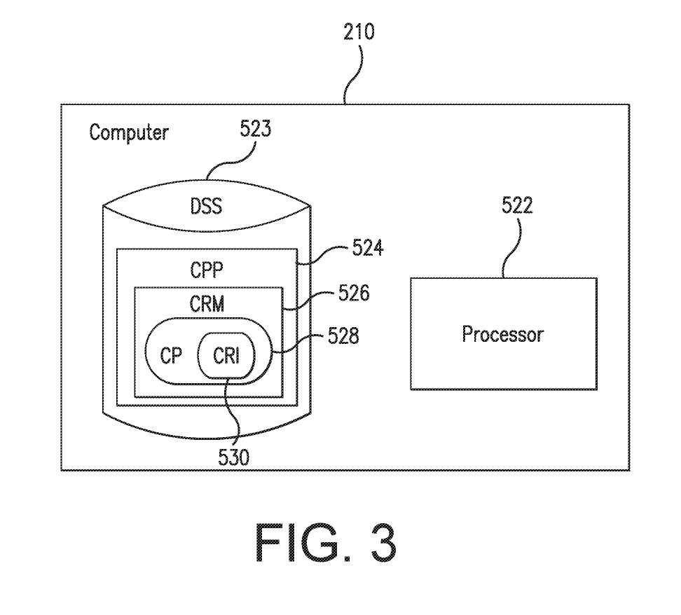

FIG. 3 is a block diagram of a non-limiting embodiment of the computer 210 of the analyte monitoring system 50. As shown in FIG. 3, in some embodiments, the computer 210 may include one or more processors 522 (e.g., a general purpose microprocessor) and/or one or more circuits, such as an application specific integrated circuit (ASIC), field-programmable gate arrays (FPGAs), a logic circuit, and the like. In some embodiments, the computer 210 may include a data storage system (DSS) 523. The DSS 523 may include one or more non-volatile storage devices and/or one or more volatile storage devices (e.g., random access memory (RAM)). In embodiments where the computer 210 includes a processor 522, the DSS 523 may include a computer program product (CPP) 524. CPP 524 may include or be a computer readable medium (CRM) 526. The CRM 526 may store a computer program (CP) 528 comprising computer readable instructions (CRI) 530. In some embodiments, the CRM 526 may store, among other programs, the MMA, and the CRI 530 may include one or more instructions of the MMA. The CRM 526 may be a non-transitory computer readable medium, such as, but not limited, to magnetic media (e.g., a hard disk), optical media (e.g., a DVD), solid state devices (e.g., random access memory (RAM) or flash memory), and the like. In some embodiments, the CRI 530 of computer program 528 may be configured such that when executed by processor 522, the CRI 530 causes the computer 210 to perform steps described below (e.g., steps described below with reference to the MMA). In other embodiments, the computer 210 may be configured to perform steps described herein without the need for a computer program. That is, for example, the computer 210 may consist merely of one or more ASICs. Hence, the features of the embodiments described herein may be implemented in hardware and/or software.

In some embodiments in which the user interface 240 of the display device 105 includes the display 218, the MMA may cause the display device 105 to provide a series of graphical control elements or widgets in the user interface 240, such as a graphical user interface (GUI), shown on the display 218. The MMA may, for example without limitation, cause the display device 105 to display analyte related information in a GUI such as, but not limited to: one or more of analyte information, current analyte concentrations, past analyte concentrations, predicted analyte concentrations, user notifications, analyte status alerts and alarms, trend graphs, arrows, and user-entered events. In some embodiments, the MMA may provide one or more graphical control elements that may allow a user to manipulate aspects of the one or more display screens. Although aspects of the MMA are illustrated and described in the context of glucose monitoring system embodiments, this is not required, and, in some alternative embodiments, the MMA may be employed in other types of analyte monitoring systems.

In some embodiments where the display device 105 communicates with a transceiver 101, which in turn obtains sensor measurement data from the analyte sensor 100, the MMA may cause the display device 105 to receive and display one or more of glucose data, trends, graphs, alarms, and alerts from the transceiver 101. In some embodiments, the MMA may store glucose level history and statistics for a patient on the display device 105 (e.g., in memory 214 and/or DSS 533) and/or in a remote data storage system.

In some embodiments, a user of the display device 105, which may be the same or different individual as patient, may initiate the download of the MMA from a central repository over a wireless cellular network or packet-switched network, such as the Internet. Different versions of the MMA may be provided to work with different commercial operating systems, such as the Android OS or Apple OS running on commercial smart phones, tablets, and the like. For example, where display device 105 is an Apple iPhone, the user may cause the display device 105 to access the Apple iTunes store to download a MMA compatible with the Apple OS, whereas where the display device 105 is an Android mobile device, the user may cause the display device 105 to access the Android App Store to download a MMA compatible with the Android OS.

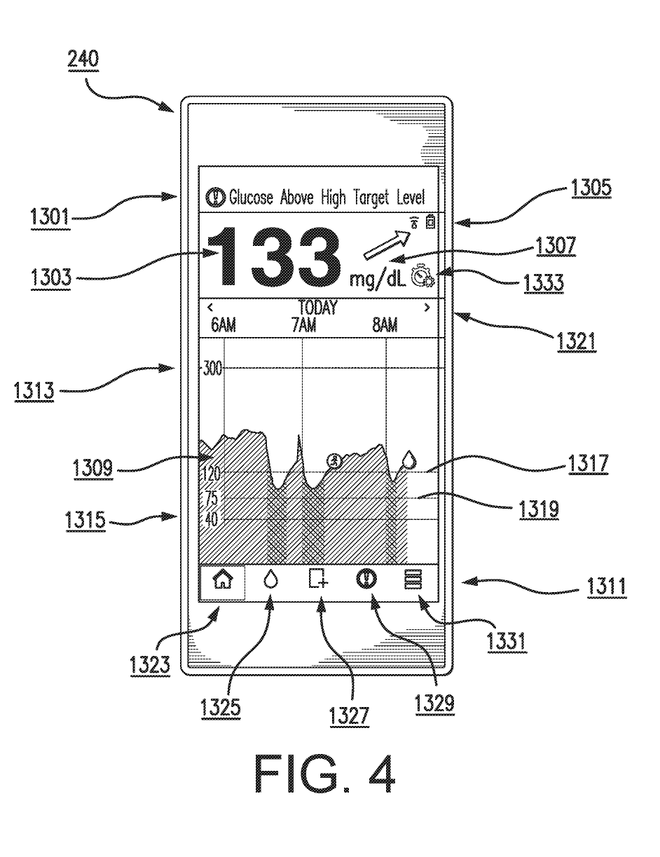

FIG. 4 is an example of a home screen display of a medical mobile application (MMA) in accordance with aspects of various embodiments of the present invention. According to some embodiments, the workspace display of the MMA may be depicted in a GUI on the display 220 of the display device 105. In some embodiments, the home screen may display one or more of real-time analyte concentrations received from transceiver 101, rate and direction of analyte level change, graphical trends of analyte levels, alarms or alerts for hypoglycemia or hyperglycemia, and logged events such as, for example and without limitation, meals, exercise, and medications. Table 1 below depicts several informational non-limiting examples of items and features that may be depicted on the home screen.

TABLE 1
Home Screen

| Status bar | Shows the status of user's glucose level |
|---|---|
| Transceiver/Transmitter | This is the transceiver being used; the transceiver name |
| ID | can be changed by going to Settings > System |
| Current glucose value | A real-time glucose reading; this may be updated every 5 minutes |
| Date and time | The current date and time with navigational options, such as scroll |
| | left or right to see different dates and times |
| Alarm and Events | Shows an icon when an alert, alarm, or event occurs |
| Bluetooth Connection | Shows the strength of the Bluetooth connection |
| Handheld Device Battery | Indicates the battery strength of the handheld device |
| Level | |
| Transmitter/Transceiver | Indicates the battery strength of the transceiver |
| Battery Level | |
| Transmitter/Transceiver | Shows the strength of the transceiver connection |
| Connection Status Icon | |
| Trend Arrow | Shows the direction a patient's glucose level is trending |
| Unit of Measurement | This is the units for the glucose value |
| High Glucose Alarm | This is the high glucose alarm or alert level set by a user |
| Level | |
| Glucose High Target | This is the high glucose target level set by a user |
| Level | |
| Stacked Alerts | Shows when there are several alerts at the same time |
| Glucose Trend Graph | A user can navigate or scroll through the graph to see the trend |
| | over time |
| | Navigation to various sections of the MMA, such as: |

| | | | |
|---|---|---|---|
| Menu | Home | Reports | Settings |
| | Calibrate | Share My Data | About |
| | Notifications | Placement Guide | |
| | Event Log | Connect | |

| | |
|---|---|
| Calibration Point Icon | This icon appears when a calibration is entered |
| Profile Indicator | This indicator may indicate what profile is being applied, such as a |
| | normal profile, temporary profile, vacation profile, and the like. |

In some embodiments, as shown in FIG. 4, the home screen may include one or more of a status notification bar 1301, a real-time current glucose level 1303 of a patient, one or more icons 1305, a trend arrow 1307, a historical graph 1309, a profile indicator 1333, and navigation tools 1311. The status notification bar 1301 may depict, for example and without limitation, alarms, alerts, and notifications related to, for example, glucose levels and system statistics and/or status. The one or more icons 1305 may represent the signal strength of the transceiver 101 and/or the battery level of the transceiver 101. The trend arrow 1307 may indicate a rate and/or direction of change in glucose measurements of a patient. The historical graph may be, for example and without limitation, a line graph and may indicate trends of glucose measurement levels of a patient. The navigation tools 1311 may allow a user to navigate through different areas or screens of the MMA. The screens may include, for example and without limitation, one or more of Home, Calibrate, Event Log, Notifications, and Menu screens.

In some embodiments, the historical graph 1309 may depict logged events and/or user inputted activities such as meals (nutrition, amount of carbohydrates), exercise (amount of exercise), medication (amount of insulin units), and blood glucose values as icons on positions of the graph corresponding to when such events occurred. In some embodiments, the historical graph 1309 may show one or more of a boundary or indication of a high glucose alarm level 1313, a low glucose alarm level 1315, a high glucose target level 1317, and a low glucose target level 1319. In some embodiments, a user may interact with a time or date range 1321 option via the GUI to adjust the time period of the glucose level displayed on the historical graph 1309. In some embodiments, the date range 1321 may be specified by a user and may bet set to different time periods such as 1, 3, 24 hours, 1, 7, 14, 30, and 60 days, weeks, months, etc. In some embodiments, the line graph 1309 may show high, low, and average glucose levels of a patient for the selected date range 1321. In other embodiments, the line graph 1309 may be a pie chart, log book, modal day, or other depiction of glucose levels of a patient over a selectable date range 1321, any of which may further depict high, low, and average glucose levels of the patient over that date range 1321.

In some non-limiting embodiments, the trend arrow 1307 may be depicted in five different configurations that signify direction (up, down, neutral) and rate (rapidly, very rapidly slow, slow, very slow, and stable) of glucose change. In some non-limiting embodiments, the MMA and/or the transceiver 101 may use the last twenty minutes of continuous glucose measurement data received from the sensor 101 and/or processed by the transceiver 730 in the calculation used to determine the orientation of the trend arrow 1307. In some embodiments, there may be times when the trend arrow 1307 may not be displayed due to, for example, there being insufficient sensor values available for the trend calculation. In some embodiments, a trend arrow 1307 displayed in a horizontal orientation (approximately 0° along the horizontal direction of the GUI display) may indicate that the glucose level is changing gradually, such as, for example, at a rate between −1.0 mg/dL and 1.0 mg/dL per minute. In some embodiments, a trend arrow 1307 displayed slightly in the upwards direction (approximately 45° up from the horizontal direction of the GUI display) may indicate that the glucose level is rising moderately, such as, for example, at a rate between 1.0 mg/dL and 2.0 mg/dL per minute. In some embodiments, a trend arrow 1307 displayed slightly in the downwards direction (approximately 45° down from the horizontal direction of the GUI display) may indicate that the glucose level is falling moderately, such as, for example, at a rate between 1.0 mg/dL and 2.0 mg/dL per minute. In some embodiments, a trend arrow 1307 displayed in a vertical direction (approximately 90° up from the horizontal direction of the GUI display) may indicate that the glucose level is rising very rapidly, such as, for example, at a rate more than 2.0 mg/dL per minute. In some embodiments, a trend arrow 1307 displayed in a downwards direction (approximately 90° down from the horizontal direction of the GUI display) may indicate that the glucose level is falling very rapidly, such as, for example, at a rate more than 2.0 mg/dL per minute. In some embodiments, the trend arrow 1307 is different from a predicted glucose alarm or alert. For example, the trend arrow 1307 may indicate rate and direction of change regardless of glucose value, whereas predicted glucose alarms or alerts may indicate reaching a certain glucose level based on current trends. For example, the MMA may cause a predicted low glucose alarm or alert to be displayed in the notification bar 1301 while still displaying a relatively stable trend arrow 1307 (e.g., at 0° or 45° from the horizontal direction of the GUI display).

In some embodiments, the historical line graph 1309 may allow user to quickly review and analyze historical data and/or trend information of a patient's glucose levels over time. In some embodiments, the historical line graph 1309 may include icons or markers along the trend line to reflect alarms, alerts, notifications, and/or any events that were automatically or manually logged by the user into the display device 105 via a GUI display generated by the MMA. Where one or more of such icons or markers are displayed on the historical line graph 1309, a user may select any one of the icons or markers to obtain more information about the item. For example, in response to a selection of a mark on the line graph 1309, the MMA may generate a popup window on the display 220 that provides more information about the mark.

In some embodiments, the historical line graph 1309 may enable a user to quickly review how well a patient is doing against glucose targets and/or alarms or alerts. For example, a user may establish a high glucose alarm level 1313 and/or a low glucose alarm level 1315, as well as a high glucose target level 1317 and/or a low glucose target level 1319. The high glucose alarm level 1313 and/or low glucose alarm level 1315 may be visually depicted over the historical line graph 1309, for example, using a colored dashed line (such as red). Additionally, the high glucose target level 1317 and low glucose target level 1319 may be visually depicted over the historical line graph 1309, for example, using a color dashed line (such as green).

In some embodiments, the colors of the historical line graph 1309 may change depending on a glucose level 1303 status. For example, during the times where the glucose level 1303 was outside of the high glucose alarm level 1313 or low glucose alarm level 1315, then the portion of the line graph 1309 corresponding to those times may be filled in red. As another example, during the times where the glucose level 1303 is between the high glucose target level 1317 and the low glucose target level 1319, then the portion of the line graph 1309 corresponding to those times may be filled in green. As yet another example, during the times where the glucose level 1303 is between a glucose target level 1317, 1319 and a corresponding alarm level 1313, 1315, then the portion of the line graph 1309 may be filled in yellow.

In some embodiments, the line graph 1309 may be displayed with one or more selectable date range icons 1321 that allow a user to change the day/time period corresponding to the line graph 1309 in real-time. For example, a user may select a forwards or backwards selectable option (such as an arrow) or use a swipe or fling gesture that may be recognized by GUI to navigate to a later or earlier time period, respectively, such as a day, month, etc. In some embodiments a user may choose an older graph 1309 to display by tapping the date on the date range 1321 portion of the screen and submitting or entering a desired date and/or time to review. In some embodiments, a user may use one or more gestures that are recognized by the GUI, such as a pinch, zoom, tap, press and hold, or swipe, on graph 1309. For example, a user may pinch the historical line graph 1309 with a thumb and index finger in order to cause the MMA to display different time/dating settings or adjust a time/date setting on the line graph 1309. In some embodiments, a user may tap or press and hold a time event on historical line graph 1309, and in response the MMA may display further detail on the time event, such as a history, reading value, date/time, or association to other events or display a prompt for entry of a time event.

In some embodiments, the MMA may store glucose data 1303 on the display device 105 (e.g., in memory 214 and/or DSS 533) so long as there is available memory space. Additionally or alternatively, the MMA may cause the display device 105 to send a sync request message to store the glucose data 1303 on a remote storage device.

In some embodiments, the MMA may cause the GUI to display navigational tools 1311 that allow a user to navigate to different features and screens provided by the MMA. For example, the navigational tools 1311 may comprise a navigation bar with one or more of a plurality of selectable navigation options 1323, 1325, 1327, 1329, and 1331, such as buttons or icons. As shown in FIG. 4, in some embodiments, the selectable navigation options may allow a user to navigate to one or more of the “Home” screen 1323, a “Calibrate” screen 1325, an “Event Log” screen 1327, a “Notifications” screen 1329, and a “Menu” screen 1331. Upon a user selection of one of the selectable navigation options in the navigation tools area 1311, a new screen corresponding to the selected option may be displayed on a display device by the GUI.

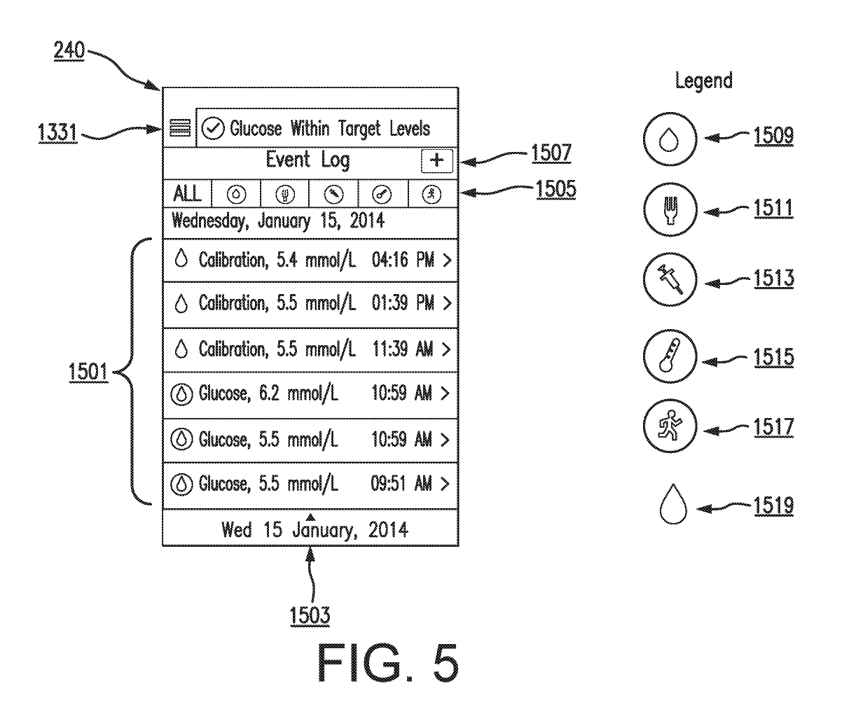

FIG. 5 is an example event screen display of a medical mobile application (MMA) in accordance with aspects of various embodiments of the present invention. In some embodiments, a user may navigate to the event screen by selecting an “Event Log” navigational tool 1327 or by using a menu navigation tool 1331 as shown in FIG. 4. In some embodiments, the MMA may cause the GUI to display the menu navigation tool or selectable option 1331 at the upper left corner of the display 220 of the display device 105 in addition to or in alternative to displaying the menu selectable option on the bottom of the display 220 with additional navigation tools 1311. The system may allow a user to log and track a plurality of events in addition to continually monitoring glucose levels of a patient. A user may manually enter events, which may appear on the trend graph 1309 and/or in any glucose reports. Such events may assist a user/patient in finding patterns in a glucose profile of a patient.

As shown in FIG. 5, the event screen may depict an event log 1501 with one or more past events. The event log 1501 may list all or a subset of events entered by a user over a specific time period (e.g., a day, a week, a month, a year, etc.). Where the events in the event log 1501 span beyond the display area of the display device 105, the MMA may configure the GUI to allow a user to use a gesture, such as a scroll or flick, that is recognized by the GUI to navigate through the event entries and/or configure the GUI to provide a selectable date option 1503 to allow a user to navigate to a different date of events. In some embodiments, each event in the event log 1501 may be selectable in the GUI, and upon selection of the event a screen may appear indicating one or more additional details of the event, such as the time, notes, values, measurements, etc. associated with the event.

Each event may correspond to an event type, which may be represented in short-hand with a symbol and/or a specific icon such as those shown in FIG. 5. For example, icon 1509 may indicate a BGM test event, icon 1511 may indicate a meal event, icon 1513 may indicate an insulin dosage event, icon 1515 may indicate a health condition event, icon 1517 may indicate an exercise event, and icon 1519 may indicate a calibration measurement. In some embodiments, different icons may be used to indicate the same or different events as those shown in the legend.

Where the MMA specifies event types, such as those shown in the legend in FIG. 5, the event screen may display a set of one or more selectable filtering options 1505 to filter the types of events displayed in the event log 1501. When the “all” events option is selected by a user, all events regardless of type may be displayed in the event log 1501. However, upon the selection of a selectable event filtering option 1505 by a user, which may be represented as one or more icons, such as those shown in the legend, only events corresponding to the selected filtering option 1505 may be displayed. For example, if a user selects the icon 1509 corresponding to BGM tests, then the MMA may only display events, if any, that reflect BGM tests in the event log 1501.

In some embodiments, a user may manually add event entries by selecting an add event option 1507. Upon selection by a user of the add event option 1507, the MMA may cause the GUI to prompt the user to specify the type of the event, such as, for example, a BGM test event, a meal event, an insulin dosage event, a health condition event, an exercise event, or the like. After selection by the user of the type of event, the MMA may cause the GUI to display one or more parameters associated with the event. For example, where a patient and/or user takes a blood glucose test outside of a calibration measurement window and wishes to simply log the measurement, the user may select the add event option 1507, select “Glucose” to specify the type of event, and then enter parameters into the GUI associated with the event, such as time, date, glucose value, notes and the like. As another example, for a meal event, a user may enter parameters associated with the event such as time, date, type of meal, carbohydrates, and any notes. As another example, for an insulin dosage event, a user may enter parameters associated with the event such as time, date, units, type of insulin, and notes. As another example, for a health event, a user may enter parameters associated with the event such as time, date, severity (low, medium, high), condition, and notes. As yet another example, for an exercise event, a user may enter parameters associated with the event such as time, date, intensity (low, medium, high), duration, and notes.

In some embodiments, the user may be presented with an option to “select from a database” of activity. For example, the user may be able to indicate intensity level and duration associated with the activity item, and the activity item may be used in, for example, the event log.

In some embodiments, one or more different event types may be predefined by the MMA. In some embodiments, a user may be able to customize or define a new event type in addition to or in lieu of any MMA predefined event types.

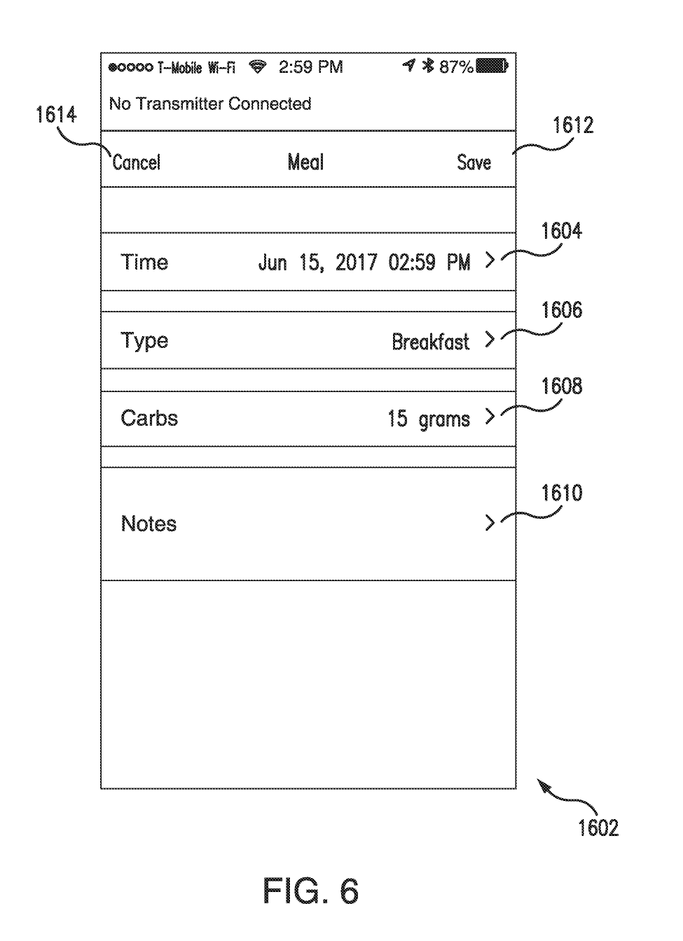

FIG. 6 illustrates a non-limiting example of a meal event entry screen 1602 displayed on the display 220 of the user interface 240 of the display device 105 according to some embodiments of the present invention. In some embodiments, the MMA may display the meal entry screen on the display 220 of the user interface 240 after the user selects the add event option 1507 and then selects “Meal” to specify the type of event. In some embodiments, as shown in FIG. 6, the meal event entry screen 1602 may include one or more of a selectable element 1604 for specifying a time and date, a selectable element 1606 for specifying a type of meal, a selectable element 1608 for specifying an amount of carbohydrates, and a selectable element 1610 for adding notes. In some embodiments, upon selection of the notes selectable element 1610, the user may be able to enter notes about the meal event. In some embodiments, the meal event entry screen 1602 may include a cancel event entry selectable element 1614 and a save event entry selectable element 1612.

In some embodiments, the MMA may include a default time, and the meal event entry screen 1602 may be initially set to and display the default time and date. In some non-limiting embodiments, the default time and date may be the current time and date. In some embodiments, selecting the specify time and date selectable element 1604 may enable the user to adjust and/or specify the time at which the patient consumed the meal (i.e., the time at which the meal event occurred).

In some embodiments, the MMA may include a default amount of carbohydrates, and the meal event entry screen 1602 may be initially set to and display the default amount of carbohydrates. In some non-limiting embodiments, the default amount of carbohydrates may be a number of calories (e.g., 300 calories) or a portion size, weight, or mass (e.g., 15 grams). In some embodiments, selecting the specify amount of carbohydrates selectable element 1608 may enable the user to adjust and/or specify the amount of carbohydrates that the patient consumed during the meal event. In some embodiments, when entering a meal event, the user may be presented with an option to “select from a database” of foods. For example, the user may be able to indicate portion size as a multiplier, and carbohydrate values associated with a selected food item from the database of foods may be used in an event log. In some embodiments, the user may be able to select multiple food items from the food database to compose one “meal” associated with a meal event entry, and the total carbohydrate value for the meal may be used in, for example, the event log.

In some embodiments, the MMA may include a default meal type, and the meal event entry screen 1602 may be initially set to and display the default meal type (e.g., breakfast). In some embodiments, selecting the specify type of meal selectable element 1606 may enable the user to adjust and/or specify the type of meal consumed by the patient during the meal event. In some non-limiting embodiments, upon selecting the specify type of meal selectable element 1606, the user may select from one or more of the following meal types: breakfast, lunch, dinner, and snack.

In embodiments having a static default meal type, if the user want to enter a meal type that is different than the default meal type, the user may select the specify type of meal selectable element 1606 and then select the proper meal type, which may require an extra click or two depending on the meal type. Accordingly, in some embodiments, the MMA have a dynamic default meal type. In some non-limiting embodiments, the MMA may select the default meal type based on the current time of day. For instance, in some non-limiting embodiments, the MMA may select the meal type that is appropriate for the current time of day as the default meal type to display in the meal event entry screen 1602. In some non-limiting embodiments, the MMA may select breakfast as the default meal time in the mornings (e.g., from 5:00 am to 10:00 am), lunch as the default meal type around noon (e.g., from 11:00 am to 2:00 pm), and dinner as the default meal type in the evenings (e.g., from 4:30 pm to 10:00 pm). In some embodiments, the MMA may select snack as the default meal type during one or more time periods (e.g., from 10:00 am to 11:00 am and/or from 2:00 pm to 4:30 pm) or during any time not assigned to breakfast, lunch, or dinner. In some embodiments, the dynamic default meal type may simplify and/or accelerate entry of a meal type (e.g., because meal event entry with the dynamic default meal type will require fewer clicks than meal event entry with a static default meal type).

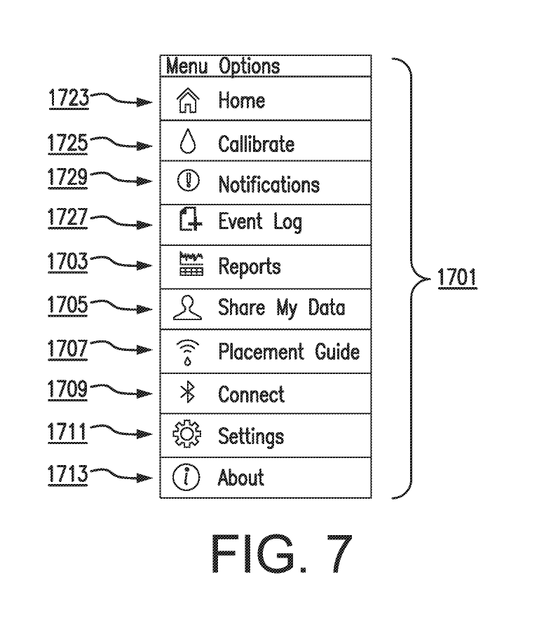

FIG. 7 is an example menu navigational bar screen display of a medical mobile application in accordance with aspects of various embodiments of the present invention. As described above, in some embodiments the MMA home screen may include interactive navigational tools including a menu navigational bar 1329 with a selectable menu option 1331. Additionally, or in the alternative, a selectable menu option 1331 may persist in specific location across multiple screens generated by the MMA and displayed in the GUI, such as in the top left corner of the display 220 or another location of the display 220. On selection of the selectable menu option 1331 by a user, a menu bar 1701 of one or more selectable options may be displayed by the MMA on the GUI. For example, as shown in FIG. 6, the one or more selectable options may correspond to a home or home screen 1723 (e.g., as shown in FIG. 4), a calibrate screen 1725, a notification screen 1729, an event screen 1727 (e.g., as shown in FIG. 5), a reports screen 1703, a share my data screen 1705, a placement guide 1707, a connect screen 1709, a settings screen 1711, and an about screen 1713. In response to a selection of one of the selectable options in menu bar 1701, the MMA may display one or more screens associated with the selectable options.

As described above, the home screen corresponding to selectable option 1723 may be a main screen with glucose information for a patient, including current glucose level, trends, status, and/or graph information. The calibrate screen corresponding to selectable option 1725 may be a screen where a user can submit a calibration BGM measurement value. The notification screen corresponding to selectable option 1729 may correspond to a display of a list of past notifications, alerts, and alarms. The event log screen corresponding to selectable option 1727 may correspond to a display of a list of events such as meals, insulin, and exercise, of a patient and provide an option for a user to submit a new event.

In some embodiments, the reports selectable option 1703 may cause the MMA to configure the GUI to display one or more screens that allow a user to view pre-formatted reports based on glucose data. In some embodiments, one or more of the following types of reports may be selected by the MMA and/or a user to be displayed: a weekly summary report with a seven-day summary graph and statistics; a modal day with a graphical view of continuous glucose readings over several days displayed in a 24-hour timeline; statistics; glucose distribution; and a logbook.

In some embodiments, the share my data selectable option 1705 may cause the MMA to display one or more screens that allow a user to share reports and other information with others via email or to another display device 105. In some embodiments, the MMA may include a “share my data” setting that enable or disable sharing of patient information with other individuals. For example, the MMA may maintain a list of one or more members with whom data may be shared and their associated contact information, such as email addresses, telephone number, social media account. If the “share my data” setting is enabled, the MMA may cause the display device 105 (e.g., the second wireless communication IC 216 and/or the connector IC 204) to transmit shared information over a wireless and/or wired communication link using, for example, one or more simple mail transfer protocol (SMTP) messages, short message service (SMS) messages, social media (e.g., Twitter) messages, enhanced messaging service (EMS) messages, or telephonic messages. For example, the MMA may cause the display device 105 to transmit shared information via one or more SMTP messages to the email addresses corresponding to the list of members. In some embodiments, members may include one or more of a caregiver, physician, or family member. In some embodiments, the MMA may allow the sharing of glucose reports with up to five people, or more. In some embodiments, the MMA may allow a user to share CGM data, such as glucose and trend graph and/or CGM notifications, alerts, and alarms.

In some embodiments, the settings selectable option 1711 may cause the MMA to display one or more screens in the GUI that allow a user to customize settings such as alarms, alerts, calibration schedule, and system information. In some embodiments, customization of the settings may better help create a glucose profile that fits a patient's needs. There may be four areas where the MMA may provide customization, including: 1) glucose settings—glucose levels and rates that will set an alarm or alert (audible or vibratory) once the level or rate is crossed; 2) daily calibration settings—the morning and afternoon calibration reminder in the daily calibration phase; 3) system settings—identifies or sets various system-related information; and 4) mealtimes settings—designated times for meals so as to format glucose reports

In some embodiments, the MMA may provide default time intervals for regular hours for one or more of breakfast, lunch, snack, and dinner meal events. In some embodiments, a user may adjust these default time intervals for one or more of the breakfast, lunch, snack, and dinner meal events. In some embodiments, the time intervals corresponding to the meal times may be utilized on a reports graph to indicate the high, low, and average sensor glucose values during each event.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention. For instance, in some non-limiting embodiments, the transceiver 101 may be a smartphone (e.g., an NFC-enabled smartphone). In some non-limiting embodiments, a smartphone (e.g., an NFC-enabled smartphone) may be used in place of the transceiver 101 and the display device 105. That is, in some alternative embodiments, a smartphone may be used to communicate directly with the sensor 100, power the sensor 100, calculate glucose concentrations using sensor data received from the sensor 100, and execute the MMA, which that displays the glucose concentrations and/or other analyte monitoring information (e.g., analyte trends, alerts, alarms, notifications). In some of these alternative embodiments, the smartphone may include the elements illustrated in FIGS. 2 and 3, and the smartphone may additionally include a sensor interface element that enables direct communication with the analyte sensor 100. The sensor interface may include, for example and without limitation, one or more of an inductive element, an RFID reader IC, and a power amplifier, such as those described with reference to FIG. 5 of U.S. patent application Ser. No. 15/786,954, filed on Oct. 18, 2017, which is incorporated herein by reference in its entirety.

What is claimed is:

1. An analyte monitoring system comprising:
an analyte sensor;
a transceiver configured to (i) receive measurement information from the analyte sensor, (ii) calculate one or more analyte concentrations using at least the received measurement information, and (iii) convey the calculated one or more analyte concentrations; and
a display device configured to (i) receive the one or more analyte concentrations from the transceiver, (ii) display the received one or more analyte concentrations, (iii) select one of a plurality of meal types as a default meal type based on the current time of day, and (iv) display a meal event entry screen that includes a meal type selectable element for specifying a type of meal, wherein the meal type selectable element is initially set to the selected default meal type, and the meal type selectable element is capable of being set to a different one of the plurality of meal types.

2. The system of claim 1, wherein the display device is configured to display a meal event entered using the meal event entry screen with the one or more analyte concentrations.

3. The system of claim 1, wherein the plurality of meal types include breakfast, lunch, and dinner.

4. The system of claim 1, wherein selecting the default meal type comprises comparing the current time of day to time intervals corresponding to the meal types, and the selected default meal type is the meal type having the time interval into which the current time of day falls.

5. The system of claim 4, wherein the time intervals are configured to be adjustable by a user of the display device.

6. An analyte monitoring system comprising:
an analyte sensor;
a transceiver configured to (i) receive measurement information from the analyte sensor, (ii) calculate one or more analyte concentrations using at least the received measurement information, (iii) display the calculated one or more analyte concentrations, (iv) select one of a plurality of meal types as a default meal type based on the current time of day, and (v) display a meal event entry screen that includes a meal type selectable element for specifying a type of meal, wherein the meal type selectable element is initially set to the selected default meal type, and the meal type selectable element is capable of being set to a different one of the plurality of meal types.

7. The system of claim 6, wherein the transceiver is configured to display a meal event entered using the meal event entry screen with the one or more analyte concentrations.

8. The system of claim 6, wherein the plurality of meal types include breakfast, lunch, and dinner.

9. The system of claim 6, wherein selecting the default meal type comprises comparing the current time of day to time intervals corresponding to the meal types, and the selected default meal type is the meal type having the time interval into which the current time of day falls.

10. The system of claim 9, wherein the time intervals are configured to be adjustable by a user of the transceiver.

11. A display device comprising:
a transceiver interface device configured to receive one or more analyte concentrations from a transceiver;
a user interface;
a computer including a non-transitory memory and a processor, wherein the computer is configured to:
cause the user interface to display the one or more received analyte concentrations;
select one of a plurality of meal types as a default meal type based on the current time of day;
cause the user interface to display a meal event entry screen that includes a meal type selectable element for specifying a type of meal, wherein the meal type selectable element is initially set to the selected default meal type, and the meal type selectable element is capable of being set to a different one of the plurality of meal types.

12. The display device of claim 11, wherein the computer is configured to cause the user interface to display a meal event entered using the meal event entry screen with the one or more analyte concentrations.

13. The display device of claim 11, wherein the plurality of meal types include breakfast, lunch, and dinner.

14. The display device of claim 5, wherein selecting the default meal type comprises comparing the current time of day to time intervals corresponding to the meal types, and the selected default meal type is the meal type having the time interval into which the current time of day falls.

15. The display device of claim 14, wherein the time intervals are configured to be adjustable by a user of the display device.

16. A transceiver comprising:
a sensor interface device configured to receive sensor data from an analyte sensor;
a user interface;
a computer including a non-transitory memory and a processor, wherein the computer is configured to:
calculate one or more analyte concentrations using at least the received measurement information;
cause the user interface to display the one or more calculated analyte concentrations;
select one of a plurality of meal types as a default meal type based on the current time of day; and
cause the user interface to display a meal event entry screen that includes a meal type selectable element for specifying a type of meal, wherein the meal type selectable element is initially set to the selected default meal type, and the meal type selectable element is capable of being set to a different one of the plurality of meal types.

17. The transceiver of claim 16, wherein the computer is configured to cause the user interface to display a meal event entered using the meal event entry screen with the one or more analyte concentrations.

18. The display device of claim 16, wherein the plurality of meal types include breakfast, lunch, and dinner.

19. The display device of claim 16, wherein selecting the default meal type comprises comparing the current time of day to time intervals corresponding to the meal types, and the selected default meal type is the meal type having the time interval into which the current time of day falls.

20. The display device of claim 19, wherein the time intervals are configured to be adjustable by a user of the transceiver.

21. An analyte monitoring system comprising:
an analyte sensor;
a transceiver configured to (i) receive measurement information from the analyte sensor, (ii) calculate one or more analyte concentrations using at least the received measurement information, and (iii) convey the calculated one or more analyte concentrations; and
a display device configured to (i) receive the one or more analyte concentrations from the transceiver, (ii) display the one or more received analyte concentrations, (iii) enable a user to adjust default time intervals for meal types of a plurality of meal types, and (iv) display an event log screen that includes an add event selectable element for adding one or more events including one or more meal events based on the adjusted default time intervals.

22. The system of claim 21, wherein the plurality of meal types include breakfast, lunch, and dinner.

23. A display device comprising:
a transceiver interface device configured to receive one or more analyte concentrations from a transceiver;
a user interface;
a computer including a non-transitory memory and a processor, wherein the computer is configured to:
cause the user interface to display the one or more received analyte concentrations;
cause the user interface to enable a user to adjust default time intervals for meal types of a plurality of meal types;
cause the user interface to display an event log screen that includes an add event selectable element for adding one or more events including one or more meal events based on the adjusted default time intervals.

24. The display device of claim 23, wherein the plurality of meal types include breakfast, lunch, and dinner.
